(19) United States
(12) Patent Application Publication
(10) Pub. No.: US 2025/0152550 A1
(43) Pub. Date: May 15, 2025

(54) LYMPH-TARGETING FORMULATIONS

(71) Applicants: Monash University, Clayton (AU); Auckland University, Auckland (NZ)

(72) Inventors: Natalie TREVASKIS, Clayton (AU); Christopher John PORTER, Clayton (AU); Given LEE, Clayton (AU); Sifel HAN, Clayton (AU); Anthony PHILLIPS, Auckland (NZ); John WINDSOR, Auckland (NZ); Jiwon HONG, Auckland (NZ); Ian Kenneth STYLES, Clayton (AU); Zijun LU, Clayton (AU)

(21) Appl. No.: 18/250,509

(22) PCT Filed: Oct. 26, 2021
(86) PCT No.: PCT/AU21/51242

§ 371 Date: Apr. 25, 2023

(30) Foreign Application Priority Data
Oct. 26, 2021 (AU) 2020903873

(51) Int. Cl. A61K 31/337; A61K 9/00; A61K 9/107; A61K 47/12; A61P 1/18

(52) U.S. Cl. CPC A61K 31/337; A61K 9/0053; A61K 9/107; A61K 47/12; A61P 1/18

(57) ABSTRACT

The present invention relates to pharmaceutical formulations comprising a lipase inhibitor and long-chain fatty acids. The formulation enables the lipase inhibitor to be delivered to the intestinal lymph and are thereby useful in the treatment or prevention of diseases and conditions mediated by pancreatic lipase, such as acute pancreatitis and associated syndromes.

### FIELD OF THE INVENTION

The present invention relates to pharmaceutical formulations comprising a lipase inhibitor and their use in the treatment or prevention of diseases and conditions mediated by pancreatic lipase, in particular, the treatment or prevention of acute pancreatitis and associated syndromes.

### BACKGROUND OF THE INVENTION

The pancreas produces enzymes that aid the digestion and absorption of food, including the enzyme pancreatic lipase, which digests lipids (i.e. fats). Acute pancreatitis is an inflammatory disease of the pancreas that has an unpredictable clinical course. It is one of the most common causes for hospitalization due to gastrointestinal disease (Peery, A. F., et al. Gastroenterology 2019, 156, 254-272 e211). In a US nationwide study (2002-2013) of almost 5 million patients with acute pancreatitis, the incidence increased significantly from 9.48 to 12.19 cases per 1000 hospitalizations (28%) (Brindise, E., el al. Pancreas, 2019, 48, 169-175). It accounts for over 270,000 annual hospital admissions in the US at a cost of $2.5b (Forsmark, C. E., el al. N Engl J Med, 2016, 375, 1972-1981). Acute pancreatitis has multiple aetiologies and a variable clinical course with a range of severities related to mortality risk. The mortality of moderately severe, severe and critical AP is 2.8%, 40% and 54% (Zubia-Olaskoaga, F., et al. Crit Care Med, 2016, 44, 910-917).

The most important determinant of the outcome from acute pancreatitis is progression to a systemic inflammatory response syndrome (SIRS) leading to multiple organ dysfunction syndrome (MODS) and organ failure. 20-30% of acute pancreatitis patients develop organ failure, which when persistent has a mortality risk of up to 50% (Johnson, C. D. & Abu-Hilal, M. Gut, 2004, 53, 1340-1344; Petrov, M. S., et al. Gastroenterology, 2010, 139, 813-820). Strikingly, there are no specific and effective treatments for acute pancreatitis or the specific prevention or treatment of SIRS, MODS and organ failure. The current management standard is supportive care (such as generic fluid and nutritional therapy) and managing complications as they occur.

Relatively recent findings provide compelling evidence that ‘toxic factors’ from the gut, including pancreatic lipase and lipase generated lipotoxins, enter lymph and the blood circulation to promote SIRS, MODS, organ failure and death in acute pancreatitis and other acute diseases (Mittal, A., et al. American Journal of Physiology-Gastrointestinal and Liver Physiology, 2012, 303, G969-G978; Deitch, E. A. Lymphatics in the Digestive System: Physiology, Health, and Disease, 2010, 1207, E103-E111; Fanous, M. Y., Phillips, A. J. & Windsor, J. A. JOP, 2007, 8, 374-399; Escott, A. B., Phillips, A. R. & Windsor, J. A. The Role of the Intestine and Mesenteric Lymph in the Development of Systemic Inflammation and MODS in Severe Acute Pancreatitis. The Pancreas (ed. H. G. Beger, et al.), 2018, 166-172). This ‘gut-lymph concept’ opens the potential to treat acute diseases such as pancreatitis by inactivating pancreatic lipases in the gut and lymph.

A number of inhibitors of pancreatic lipase, have been developed as anti-obesity drugs (Sternby, B., et al. Clinical Nutrition, 2002, 21, 395-402; Guerciolini, R. Journal of the International Association for the Study of Obesity, 1997, 21, S12-23). In this setting the lipase inhibitors reduce caloric intake by inhibiting the digestion and absorption of ingested fat. Examples of pancreatic lipase inhibitors include cetilistat, lipstatin and orlistat (marketed as Xenical by Roche and Alli by GlaxoSmithKline). Orlistat has also been reported to promote cell apoptosis, reduce cell growth and lymph node metastasis in mouse melanoma models when administered parenterally (Seguin, F., et al. Br J Cancer, 2012, 107, 977-987; Carvalho, M. A., et al. International Journal of Cancer, 2008, 123, 2557-2565).

The currently marketed formulations of lipase inhibitors for the treatment of obesity consist of the lipase inhibitor administered in standard dry-powder formulations. The absorption and bioavailability of the lipase inhibitor orlistat is minimal (<1% dose, Zhi, J., et al. The Journal of Clinical Pharmacology, 1996, 36, 1006-1011) after oral administration in these standard formulations in either the fed or fasted state. This is due to high first pass metabolism, binding to pancreatic lipases and capture within dietary fat droplets in the intestinal lumen that are not digested in the presence of orlistat (Zhi, J., Mulligan, T. E. & Hauptman, J. B. The Journal of Clinical Pharmacology, 1999, 39, 41-46). In healthy volunteers and human patients, the plasma concentrations of orlistat are negligible after oral administration in standard capsule formulations, with more than 97% of the dose recovered in the faeces of healthy volunteers and 83.1% in a non-metabolised form (Zhi, J., et al. The Journal of Clinical Pharmacology, 1995, 35, 1103-1108; Zhi, J., Mulligan, T. E. & Hauptman, J. B. The Journal of Clinical Pharmacology, 1999, 39, 41-46). The low absorption of orlistat after oral administration in standard formulations limits its potential to treat systemic conditions such as acute pancreatitis.

Accordingly, there exists a need to develop novel formulations and methods for the treatment of diseases and conditions mediated by pancreatic lipase including acute diseases such as acute pancreatitis and associated syndromes.

### SUMMARY OF THE INVENTION

New formulations and methods are provided to deliver lipase inhibitors to the intestinal lymph.

Accordingly, in one aspect the present invention provides a pharmaceutical formulation comprising a lipase inhibitor and one or more long chain fatty acid.

In another aspect, the present invention provides a method of treating or preventing a disease or condition mediated by pancreatic lipase in a subject in need thereof comprising administering to the subject an effective amount of the pharmaceutical formulation according to the invention.

In a further aspect, the present invention provides a method of treating or preventing acute pancreatitis or an associated syndrome of acute pancreatitis selected from systemic inflammatory response syndrome and/or multiple organ dysfunction syndrome in a subject in need thereof, comprising administering to the subject an effective amount of the pharmaceutical formulation according to the invention.

In another aspect, the present invention provides a method of treating or preventing acute pancreatitis or an associated syndrome of acute pancreatitis selected from systemic inflammatory response syndrome and/or multiple organ dysfunction syndrome in a subject in need thereof, comprising enterally administering to the subject an effective amount of a lipase inhibitor and one or more long chain fatty acid, wherein the one or more long chain fatty acid is present in an amount sufficient to enhance or promote transport of the lipase inhibitor to the intestinal lymph.

In a further aspect, the present invention provides a method of treating or preventing acute pancreatitis or an associated syndrome of acute pancreatitis selected from systemic inflammatory response syndrome and/or multiple organ dysfunction syndrome in a subject in need thereof, comprising administering to the subject a formulation comprising an effective amount of a lipase inhibitor and one or more long chain fatty acid, wherein the formulation is a self-emulsifying drug delivery system and wherein the one or more long chain fatty acid is present in the formulation in an amount sufficient to enhance or promote transport of the lipase inhibitor to the intestinal lymph.

In yet a further aspect, the present invention provides a pharmaceutical formulation according to the invention for use in the treatment or prevention of acute pancreatitis or an associated syndrome of acute pancreatitis selected from systemic inflammatory response syndrome and/or multiple organ dysfunction syndrome in a subject in need thereof.

These and other aspects of the present invention will become more apparent to the skilled addressee upon reading the following detailed description in connection with the accompanying examples and claims.

### BRIEF DESCRIPTION OF THE DRAWINGS

The invention will herein be described by way of example only with reference to the following non-limiting Figures in which:

FIG. 1 illustrates the impact of formulation lipid type on mesenteric lymph transport and plasma pharmacokinetics of orlistat in lymph cannulated rats. Panel A: Cumulative lymphatic transport of total orlistat (open and closed ring forms) over time, Panel B: Cumulative lymphatic transport of closed ring orlistat over time, Panel C: Dose-normalised lymph concentration of total orlistat (open and closed ring forms) over time, Panel D: Dose-normalised lymph concentration of closed ring orlistat over time, Panel E: Dose-normalised plasma concentration of total orlistat (mostly in the open ring form) over time, and Panel F: The ratio of lymph to plasma concentrations of total orlistat over time, in mesenteric lymph duct cannulated, anesthetised rats following intraduodenal infusion of formulations from 0 to 2 h. Data is presented as mean± SEM for oleic acid LC-FA (circle, n=5), octanoic acid MC-FA (diamond, n=4), olive oil LC-TG (square, n=4) and control lipid-free (triangle, n=3) formulations. For panel E significance applies to lipid free versus MC-FA and lipid free versus LC-TG only. Significant difference to other groups determined from one-way ANOVA: ***p≤0.001, *p≤0.05

FIG. 2 illustrates the impact of lipid dose on mesenteric lymph transport and plasma pharmacokinetics of orlistat in lymph cannulated rats. Panel A: Cumulative lymphatic transport of total orlistat (open and closed ring forms) over time, Panel B: Cumulative lymphatic transport of closed ring orlistat over time, Panel C: Dose-normalised lymph concentration of total orlistat (open and closed ring forms) over time, Panel D: Dose-normalised lymph concentration of closed ring orlistat over time, Panel E: Dose-normalised plasma concentration of total orlistat (mostly in the open ring form) over time, and Panel F: The ratio of lymph to plasma concentrations of total orlistat over time in mesenteric lymph duct cannulated, anesthetised rats following intraduodenal infusion of formulations from 0 to 2 h. Data is presented as mean±SEM for 80 mg LC-FA (open circle, n=4), 40 mg LC-FA (black circle, n=5), and control (triangle, n=3). Significant difference to other groups from one-way ANOVA: **p≤0.01, *p≤0.05.

FIG. 3 illustrates the effect of lipid type on systemic exposure of orlistat. Dose-normalised plasma concentrations of total orlistat (which was predominantly in the open ring form) over time in anesthetised, carotid artery cannulated and mesenteric lymph duct cannulated (dotted line) or lymph-intact (solid line) rats following intraduodenal infusion of formulations over 2 h. Data is presented as mean±SEM for LC-FA (open circle, n=5 for lymph cannulated and shaded circle, n=4 for lymph intact) and LC-TG (black square, n=4 for lymph cannulated and shaded square, n=3 for lymph intact). Significant difference to other groups determined from one-way ANOVA: ***p≤0.001, *p≤0.05.

FIG. 4 illustrates lymph and plasma pharmacokinetics of orlistat in lymph diverted or lymph intact sham or acute pancreatitis (AP) rats intestinally administered a blank long chain fatty acid (LC-FA), orlistat LC-FA (LC-FA+O), or orlistat lipid free formulation (LFF+O). Dose-normalised lymph concentration of total orlistat (open and closed ring forms) over time (Panel A), cumulative lymphatic transport of total orlistat (open and closed ring forms) over time (Panel B), gut-lymph concentrations of triglyceride (TG) (Panel C), cumulative TG mass transport in gut-lymph (Panel D), dose-normalised plasma concentrations of total orlistat (open and closed ring forms) (Panel E), and lymph to plasma concentration ratios of orlistat (Panel F) over time. Data is presented as mean f SEM for lymph diverted sham rats dosed with orlistat LC-FA (triangle, n=5); lymph diverted AP rats dosed with orlistat LC-FA (square, n=3); lymph diverted AP rats dosed with blank LC-FA (square, n=5); lymph intact AP rats dosed with orlistat LC-FA (square, n=5); and lymph intact AP rats dosed with orlistat LFF (diamond, n=3). Significance from unpaired t test (for comparisons between 2 groups) or from one-way ANOVA (for comparisons between 3 or more groups): *p≤0.05, **p≤0.001.

FIG. 5 illustrates L2 lung cell and HMEC-1 endothelial cell viability after incubation with control media (5% v/v FBS) or 5% v/v gut-lymph from sham (SH) or acute pancreatitis (AP) rats administered orlistat (O) in a long chain fatty acid (LC-FA) formulation or blank LC-FA formulation. Panel A-B: Cell viability for L2 cells and HMEC-1 cells, respectively. Data is presented as mean±SEM except for HMEC-1 incubated with control media (where data is presented as mean only for n=2). Replicates per group was 5 except for L2 cells incubated with control media (n=4), L2 and HMEC-1 cells incubated with lymph from AP 0.5 h p.i. (n=4) and 2.5 h p.i. (n=3), L2 and HMEC-1 cells incubated with lymph from AP+O 2.5 h p.i. (n=4), and HMEC-1 incubated in 5% FBS (n=2). Significance from one-way ANOVA: ***p≤0.001, ****p<0.0001.

FIG. 6 illustrates serum cardiac function biomarker concentrations in lymph diverted or lymph intact sham or acute pancreatitis (AP) rats administered blank long chain fatty acid (LC-FA) or orlistat LC-FA (LC-FA+O) or orlistat lipid free formulation (LFF+O). Levels of cardiac troponin I (Panel A), cardiac troponin T (Panel B), creatine kinase muscle (CKM) (Panel C), fatty acid binding protein 3 (FABP-3) (Panel D), follistatin-like protein 1 (FSTL1) (Panel E), myosin light chain 3 (MYL3) (Panel F), and tissue inhibitor of metalloproteinase-1 (TIMP-1) (Panel G) were measured in serum collected at the end of each experiment (i.e. 4 hours after commencement of formulation administration and 3.5 hours after induction of AP). Data is presented as mean±SEM. Replicates per group were 5 except for AP, lymph diverted, LC-FA+O (n=3); AP, lymph diverted, LC-FA (n=6); AP, lymph intact, LFF+O (n=3). Significance from one-way ANOVA: *p≤0.05, ** p≤0.01.

FIG. 7 illustrates orlistat solubility (mg/g) in a range of lipid excipients.

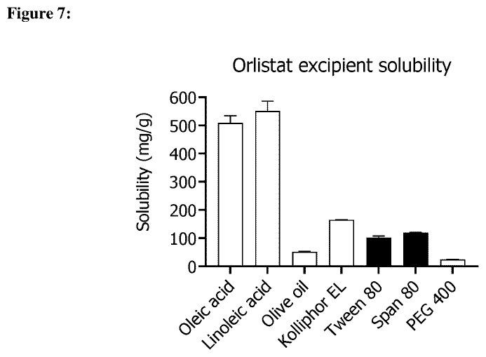

FIG. 8 illustrates (A) The mass of orlistat in the oil phase and (B) aqueous phase during the in vitro dispersion and digestion experiment for each Type IIIA formulation. Data are mean±SD (n=3).

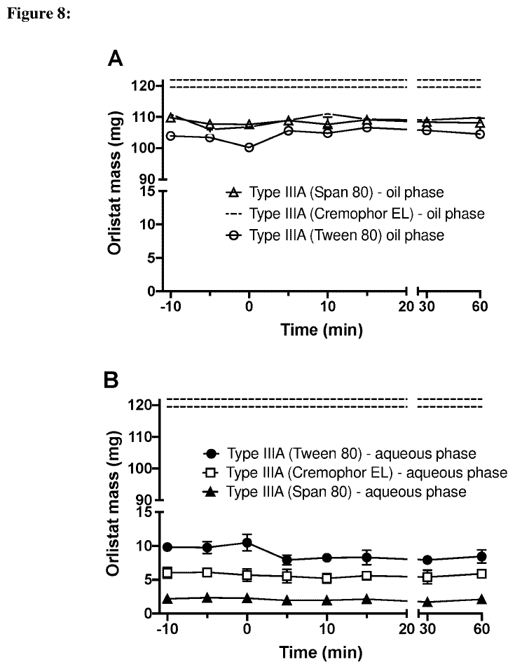

FIG. 9 illustrates mesenteric lymph concentrations of total orlistat (open and closed ring forms) in mesenteric lymph duct cannulated, anesthetised rats following intraduodenal infusion of formulations from 0 to 2 h. All formulations contained 8 mg/kg of orlistat. Data shows the lymph concentrations after administration of the Type IIIA-1 formulation (40 mg oleic acid, 50 mg P35-Eco, 10 mg PPG400) and Type IIIA-5 formulation (60 mg oleic acid, 20 mg Tween 80, 20 mg PEG400) compared to formulations in which orlistat was dispersed in 40 mg oleic acid, octanoic acid (MC-FA) or olive oil (LC-TG) with 25 mg Tween 80 and 5.6 ml PBS. Data is presented as mean±SEM for n=3-5. Dashed red line shows the IC₅₀ for orlistat inhibition of pancreatic lipase in lymph.

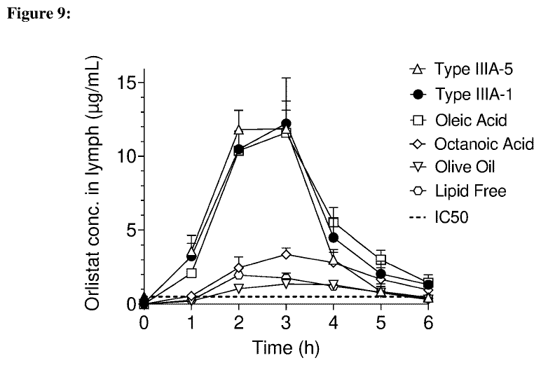

### DETAILED DESCRIPTION OF THE INVENTION

The present invention relates to pharmaceutical formulations comprising a lipase inhibitor and one or more long chain fatty acid and their use in the treatment of acute pancreatitis and associated syndromes. The invention is based, at least in part, on the discoveries that entry of pancreatic lipases and lipase generated lipotoxins into the gut-lymph and then systemic blood circulation contributes to systemic inflammatory response syndrome (SIRS), multiple organ dysfunction syndrome (MODS) and multiple organ failure in acute pancreatitis. The invention provides for an improved formulation that is able to promote absorption of lipase inhibitors from the intestine and uptake into intestinal lymph and the blood circulation.

Marketed lipid-based drug formulations for enteral or parenteral administration ordinarily consist of mixtures of glycerides, surfactants and/or co-solvents. Indeed, the lipid formulations of lipase inhibitors tested previously in experimental models of acute pancreatitis have consisted of mixture of glycerides with surfactants including natural bile salts. Lipids, such as triglycerides, use a unique metabolic pathway to gain access to the lymph (and ultimately the systemic circulation). After ingestion, dietary triglycerides are hydrolysed by luminal lipases to release one monoglyceride and two fatty acids for each molecule of triglyceride. The monoglyceride and two fatty acids are subsequently absorbed into enterocytes, where they are re-esterified to triglycerides.

Resynthesised triglycerides are assembled into intestinal lipoproteins (primarily chylomicrons) and the chylomicrons so formed are exocytosed from enterocytes and subsequently gain preferential access to the intestinal lymphatics. Within the lymphatics, lipids in the form of chylomicrons, drain through a series of capillaries, nodes and ducts, finally emptying into the systemic circulation at the junction of the left subclavian vein and internal jugular vein. Following entry into blood circulation, triglycerides in chylomicrons are preferentially and efficiently taken up by tissues with high expression of lipoprotein lipases such as adipose tissue, liver and potentially certain types of tumour tissues.

However, by their very nature, lipase inhibitors prevent digestion of glycerides and subsequent transport of lipids to the intestinal lymph. Accordingly, novel methods of transporting lipase inhibitors to the intestinal lymph are needed.

It was surprisingly found that formulating lipase inhibitors with long chain fatty acids enabled absorption without the need for lipase-mediated digestion of the formulation.

Pharmaceutical formulations comprising a lipase inhibitor and long chain fatty acids were far more efficacious than those formulated with glycerides and medium chain fatty acids. It is believed that this is because long chain fatty acids promote the formation and transport of lipoproteins into gut-lymph and may thus increase drug transport into gut-lymph (Trevaskis, N. L., et al. Pharm Res, 2013, 30, 3254-3270).

Accordingly, in one embodiment the present invention provides a pharmaceutical formulation comprising a lipase inhibitor and one or more long chain fatty acid.

As used herein, the term “long chain fatty acid” will be understood to mean a carboxylic acid with a long aliphatic chain that is either saturated or unsaturated. Typically, these long chain fatty acids comprise a straight-chain or unbranched saturated or unsaturated C₁₄ to C₂₄ carbon chain, although branched-chain fatty acids are also contemplated. The term “long chain fatty acid” does not include fatty acids that are conjugated, for example, to glycerol in the form of a diglyceride or triglyceride.

Suitable saturated long chain fatty acids include, but are not limited to, tetradecanoic (myristic) acid, pentadecanoic acid, hexadecanoic (palmitic) acid, heptadecanoic (margaric) acid, octadecanoic (stearic) acid, nonadecanoic acid, eicosanoic (arachidic) acid, heneicosanoic acid, docosanoic (behenic) acid, tricosanoic acid, tetracosanoic acid and combinations thereof.

Suitable unsaturated long chain fatty acids include, but are not limited to, palmitoleic acid, oleic acid, linoleic acid, α-linoleic acid, linolenic acid, stearidonic acid, vaccenic acid, elaidic acid, linolelaidic acid, arachidonic acid, cervonic acid, eicosapentaenoic acid, paullinic acid, gondoic acid, erucic acid, nervonic acid, mead acid, docosatetraenoic acid, docosahexaenoic acid and combinations thereof. In one embodiment, the unsaturated long chain fatty acid is oleic acid.

The long chain fatty acid must be present in the formulation in an amount sufficient to enhance or promote transport of the lipase inhibitor to the intestinal lymph. It will be appreciated that the amount may vary, for example, depending on the subject being treated and the route of administration. Suitable amounts may lie within the range of 0.5 wt % to 95 wt %, such as in the range of 1 wt % to 80 wt %, 2 wt % to 60 wt %, 3 wt % to 50 wt % and 4 wt % to 40 wt %. In one embodiment, the long chain fatty acid is present in the formulation in an amount of at least 5 wt %. In another embodiment, the long chain fatty acid is present in the formulation in an amount of at least 10 wt %, for example, in an amount of at least 15 wt %, at least 20 wt %, at least 30 wt %, at least 40 wt % or at least 50 wt %.

In an experimental model of acute pancreatitis it was surprisingly found that administration of a lipase inhibitor in the gut-lymph directing enteral formulation of the subject invention reduced indices associated with acute pancreatitis. Lipase inhibitors are generally very highly lipophilic (log P>5) and therefore are believed to associate with lipid digestion, absorption and lipoprotein assembly pathways when administered with fatty acids formulations that are the subject of this invention resulting in drug uptake into lymph in association within lipoproteins. Lipase inhibitors suitable for the pharmaceutical formulations and methods of the subject invention will be known to those of skill in the art and include, but are not limited to, cetilistat, lipstatin, orlistat, vibralactone, ebelactone, pancilin D, valilactone, esterastin and combinations thereof. In one embodiment, the lipase inhibitor is orlistat.

The present invention limits lipase associated intestinal lymph toxicity, thereby reducing the likelihood of disease progression and complications associated with acute pancreatitis and other acute critical illnesses. Accordingly, in one embodiment the present invention provides a method of treating or preventing acute pancreatitis or an associated syndrome of acute pancreatitis selected from systemic inflammatory response syndrome and/or multiple organ dysfunction syndrome in a subject in need thereof, comprising administering to the subject an effective amount of the pharmaceutical formulation according to the invention.

In another embodiment, the invention provides a pharmaceutical formulation according to the invention for use in the treatment or prevention of acute pancreatitis or an associated syndrome of acute pancreatitis selected from systemic inflammatory response syndrome and/or multiple organ dysfunction syndrome in a subject in need thereof.

As mentioned above, the long chain fatty acid must be present in an amount sufficient to enhance or promote transport of the lipase inhibitor to the intestinal lymph. Accordingly, in a further embodiment the present invention provides a method of treating or preventing acute pancreatitis or an associated syndrome of acute pancreatitis selected from systemic inflammatory response syndrome and/or multiple organ dysfunction syndrome in a subject in need thereof, comprising enterally administering to the subject an effective amount of a lipase inhibitor and a long chain fatty acid, wherein the long chain fatty acid is present in an amount sufficient to enhance or promote transport of the lipase inhibitor to the intestinal lymph.

In yet another embodiment, the invention provides use of a lipase inhibitor in the manufacture of a medicament for the treatment or prevention of acute pancreatitis or an associated syndrome of acute pancreatitis selected from systemic inflammatory response syndrome and/or multiple organ dysfunction syndrome in a subject in need thereof, wherein the lipase inhibitor is formulated with a long chain fatty acid and wherein the long chain fatty acid is present in an amount sufficient to enhance or promote transport of the lipase inhibitor to the intestinal lymph when administered.

As previously mentioned, in order to enhance or promote transport of the lipase inhibitor to the intestinal lymph when administered, the one or more long chain fatty acids may be present in an amount of at least 5 wt %, such as least 10 wt %, at least 15 wt %, at least 20 wt %, at least 30 wt %, at least 40 wt % or at least 50 wt %.

Accordingly, in a further embodiment the invention provides use of a lipase inhibitor in the manufacture of a medicament for the treatment or prevention of acute pancreatitis or an associated syndrome of acute pancreatitis selected from systemic inflammatory response syndrome and/or multiple organ dysfunction syndrome in a subject in need thereof, wherein the lipase inhibitor is formulated with a long chain fatty acid and wherein the long chain fatty acid is present in an amount of at least 5 wt %.

In one embodiment, the formulation according to the invention is prepared as a self-emulsifying drug delivery system (SEDDS), wherein the formulation is prepared as a pre-emulsion that forms an emulsion upon contact with water or buffer. In one embodiment, the SEDDS is administered to the subject in a suitable form, such as in a capsule, to form an emulsion upon contact with intestinal fluids. In another embodiment, the SEDDS is pre-mixed with buffer prior to administration to patients via a naso-jejunal or naso-gastric tube, for example. The advantages of formulations prepared as a SEDDS include storage and transport, as it avoids the need to prepare, store and transport large volume liquid emulsions. Formulations prepared as SEDDS may also avoid long-term physical and chemical stability issues associated with liquid emulsions including potential for phase separation, bacterial growth and drug hydrolysis.

The pharmaceutical formulations of the invention may also be effective in the treatment of other conditions in which lipases play a role. These conditions include metabolic syndrome, cancer and other acute and critical conditions associated with a systemic inflammatory response syndrome and/or multiple organ dysfunction syndrome including sepsis and severe sepsis from all causes including pneumonia, meningitis, liver or spleen abscess, cholecystitis, cholangitis, infected necrotizing pancreatitis, appendicitis, gastrointestinal perforation/peritonitis, leak from gastrointestinal anastomosis, fulminant ulcerative colitis, diverticulitis, pyelonephritis, osteomyelitis, post-puerperal sepsis, soft tissue infection/gangrene, neutropenic sepsis, atypical infections in immunosuppressed patients, SARS/COVID, HIV; major trauma; major burns; massive haemorrhage or shock; cardiogenic shock; and anaphylaxis shock.

Accordingly, in a further embodiment, the present invention provides a method of treating or preventing a disease or condition mediated by pancreatic lipase in a subject in need thereof, comprising administering to the subject an effective amount of the pharmaceutical formulation according to the invention.

The term “subject” is intended to include organisms such as mammals, e.g. humans, dogs, cows, horses, pigs, sheep, goats, cats, mice, rabbits, rats, and transgenic non-human animals. In one embodiment, the subject is a human, e.g. a human suffering from, at risk of suffering from, or potentially capable of suffering from acute pancreatitis or an associated syndrome. In certain embodiments, the subject is a cat or a dog, e.g. a cat or a dog suffering from, at risk of suffering from, or potentially capable of suffering from acute pancreatitis or an associated syndrome.

As used herein, a subject is “in need of” a treatment if such subject would benefit biologically, medically or in quality of life from such treatment.

As used herein, the term “a,” “an,” “the” and similar terms used in the context of the present invention, especially in the context of the claims, are to be construed to cover both the singular and plural unless otherwise indicated herein or clearly contradicted by the context.

The terms “treatment” and “treating” as used herein cover any treatment of a condition or disease in an animal, preferably a mammal, including a human and includes the treatment of acute pancreatitis and associated syndromes such as systemic inflammatory response syndrome and/or multiple organ dysfunction syndrome. It is also envisaged that the pharmaceutical formulations of the invention may be useful in the treatment of any disease or disorder which is associated with undesirable lipase activity.

The terms “prevention” and “preventing” as used herein cover the prevention or prophylaxis of a condition or disease in an animal, preferably a mammal, more preferably a human and includes preventing acute pancreatitis and associated syndromes such as systemic inflammatory response syndrome and/or multiple organ dysfunction syndrome as well as any disease or disorder which is associated with undesirable lipase activity.

The lymph-directing formulations of the invention consist of the lipase inhibitor formulated with one or more long chain fatty acid. It is believed that administration of the formulation via an enteral route would enhance access to pancreatic lipase in the gut-lumen and gut-lymph when compared to administration via parenteral routes (such as intraperitoneal administration), as have been tested previously in experimental models of acute pancreatitis (Navina, S., et al. Science Translational Medicine, 2011, 3, 107ra110; Patel, K., et al. The American Journal of Pathology, 2015, 185, 808-819; Durgampudi, C., et al. The American Journal of Pathology, 2014, 184, 1773-1784).

Accordingly, the route of administration for the pharmaceutical formulations of the present invention is intended to include enteral administration. Liquid formulations may be administered enterally via a nasogastric tube or via a stomach or oesophageal tube.

Liquid formulations may also be administered orally in the form of liquid filled capsules, drinkable formulations, syrups, elixirs and the like. In one embodiment the liquid formulation is administered orally as a SEDDS, for example, in a liquid filled capsule.

In one embodiment, the present invention provides a pharmaceutical formulation comprising a lipase inhibitor and one or more long chain fatty acid, wherein the pharmaceutical formulation is a liquid formulation and wherein the one or more long chain fatty acid is present in the formulation in an amount of at least 5 wt %, for example, least 10 wt %, at least 15 wt %, at least 20 wt %, at least 30 wt %, at least 40 wt % or at least 50 wt %.

In some embodiments, pharmaceutical formulations of the invention may contain an effective amount of a lipase inhibitor and one or more long chain fatty acids without additional surfactants, co-surfactants or co-emulsifiers, or co-solvents, that is to say, the pharmaceutical formulation will consist essentially of an effective amount of a lipase inhibitor and one or more long chain fatty acids. In other embodiments, the pharmaceutical formulation of the invention may contain an effective amount of a lipase inhibitor and one or more long chain fatty acids together with one or more water-insoluble surfactants, optionally together with one or more co-solvents. In further embodiments, the pharmaceutical formulation of the invention may contain an effective amount of a lipase inhibitor and one or more long chain fatty acids together with one or more water-soluble surfactants, optionally together with one or more co-solvents. In some embodiments, the pharmaceutical formulation contains an effective amount of a lipase inhibitor together with a mixture of long chain fatty acids, surfactant and co-solvent. In some embodiments, pharmaceutical formulation consists essentially of an effective amount of a lipase inhibitor, one or more long chain fatty acids, one or more surfactants/co-surfactants/co-emulsifiers, and/or solvents/co-solvents.

Suitable surfactants for use in the lipid formulations include propylene glycol mono- and di-esters of C₈-C₂₂ fatty acids, such as, but not limited to, propylene glycol monocaprylate, propylene glycol dicaprylate, propylene glycol monolaurate, sold under trade names such as Span 80, Capryol® 90, Labrafac® PG, Lauroglycol® FCC, sugar fatty acid esters, such as, but not limited to, sucrose palmitate, sucrose laurate, surcrose stearate; sorbitan fatty acid esters such as, but not limited to, sorbitan laurate, sorbitan palmitate, sorbitan oleate; polyoxyethylene sorbitan fatty acid esters such as, but not limited to, polysorbate 20, polysorbate 40, polysorbate 60, polysorbate 80 (Tween 80), polysorbate 85; polyoxyethylene mono- and di-fatty acid esters including, but not limited to polyoxyl 40 stearate and polyoxyl40 oleate; a mixture of polyoxyethylene mono- and di-esters of C₈-C₂₂ fatty acids and glyceryl mono-, di-, and tri-esters of C₈-C₂₂ fatty acids as sold under tradenames such as Labrasol®, Gelucire® 44/14, Gelucire® 50/13, Labrafil®; polyoxyethylene castor oils compound such as, but not limited to, polyoxyl 35 castor oil, polyoxyl 40 hydrogenated castor oil, and polyoxyl 60 hydrogenated castor oil, as are sold under tradenames such as Super Refined™ P35 Castor Oil, P35-ECO, Cremophor®/Kolliphor EL, Cremophor®/Kolliphor® RH40, Cremophor®/Kolliphor® RH60; polyoxyethylene alkyl ether including but not limited to polyoxyl 20 cetostearyl ether, and polyoxyl 10 oleyl ether; DL-.alpha.-tocopheryl polyethylene glycol succinate as may be sold under the tradename; glyceryl mono-, di-, and tri-ester; a glyceryl mono-, di-, and tri-esters of C₈-C₂₂ fatty acid; a sucrose mono-, di- and tri-ester; sodium dioctylsulfosuccinate; polyoxyethylene-polyoxypropylene copolymers such as, but not limited to poloxamer 124, poloxamer 188, poloxamer 407; polyoxyethyleneethers of C₈-C₂₂ fatty alcohols including, but not limited to polyoxyethylenelauryl alcohol, polyoxyethylenecetyl alcohol, polyoxyethylenestearyl alcohol, polyoxyethyleneoleyl alcohol as sold under tradenames such as Brij® 35, Brij® 58, Brij® 78Brij® 98, or a mixture of any two or more thereof.

A co-emulsifier, or co-surfactant, may be used in the formulation. A suitable co-emulsifier or co-surfactant may be a phosphoglyceride or a phospholipid, for example lecithin.

Suitable solvents/co-solvents include water, saline, phosphate-buffered saline (PBS), ethanol, propylene glycol, polyethylene glycol, polypropylene glycol, diethylene glycol monoethyl ether and glycerol.

A polymer may also be used in the pharmaceutical formulation to inhibit drug precipitation or to alter the rate of drug release. A range of polymers have been shown to impart these properties and are well known to those skilled in the art. Suitable polymers include hydroxypropylmethylcellulose, hydroxypropylmethylcellulose acetyl succinate, other cellulose-derived polymers such as methylcellulose; poly(meth)acrylates, such as the Eudragit series of polymers, including Eudragit E100, polyvinylpyrrolidone or others as described, for example, in Warren et al. Mol. Pharmaceutics, 2013, 10, 2823-2848.

Pharmaceutical formulations may be chosen specifically to provide for sustained release of the lipase inhibitor in the gastrointestinal (GI) tract in order to control the rate of absorption. Many different approaches may be used to achieve these ends including the use of high melting point lipids that disperse/erode slowly in the GI tract, or polymers that form a matrix that slowly erodes. These formulations may take the form of large monolithic dose forms or may be present as micro or nano-particulate matrices as described in, for example, Wilson and Crowley Controlled Release in Oral Drug Delivery, Springer, NY, ISBN 978-1-4614-1004-1 (2011) or Wise, Handbook of Pharmaceutical Controlled Release Technology, Marcel Dekker, NY, ISBN 0-82467-0369-3 (2000).

Pharmaceutical formulations may also contain materials commonly known to those skilled in the art to be included in lipid based formulations, including antioxidants, for example, butylated hydroxyanisole (BHA) or butylated hydroxytoluene (BHT) and solidifying agents such as microporous silica, for example magnesium alumino-metasilicate (Neusilin).

In addition to a lipase inhibitor and one or more long chain fatty acids, the pharmaceutical formulation may include an inert diluent or an assimilable edible carrier. The pharmaceutical formulation may be enclosed in hard or soft shell gelatin capsule, or it may be incorporated directly with the food of the diet. For oral therapeutic administration, the pharmaceutical formulation may include excipients and be administered in the form of ingestible tablets, troches, pills, capsules and the like. The amount of lipase inhibitor in such therapeutically useful formulations is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. In some embodiments, the one or more long chain fatty acids may act as the liquid carrier. In other embodiments, additional liquid carriers such as the co-solvents described herein may be added. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the pharmaceutical formulations of the invention may be incorporated into sustained-release preparations, including those that allow specific delivery of the pharmaceutical agent to specific regions of the gut.

The term “formulation” is intended to include the formulation of a lipase inhibitor and one or more long chain fatty acids together with encapsulating material as carrier, to give a capsule in which the lipase inhibitor (with or without other carrier) is surrounded by carriers.

As will be readily appreciated by those skilled in the art, the nature of the pharmaceutically acceptable carrier will depend on the nature of the condition and the mammal to be treated. It is believed that the choice of a particular carrier or delivery system could be readily determined by a person skilled in the art. In the preparation of any formulation containing the active compound care should be taken to ensure that the activity of the lipase inhibitor is not destroyed in the process and that the active compound is able to reach its site of action without being destroyed. In some circumstances it may be necessary to protect the lipase inhibitor by means known in the art, such as, for example, micro encapsulation.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the formulations.

Enteral formulations may be prepared in the form of suppositories by mixing with appropriate bases, such as emulsifying bases or water-soluble bases.

The pharmaceutical formulation of the invention may also be administered with one or more additional therapeutic agents in combination. The combination may allow for separate, sequential or simultaneous administration of the active ingredient(s) as hereinbefore described with the other active ingredient(s). The combination may be provided in the pharmaceutical formulation comprising one or more long chain fatty acids.

The term “combination”, as used herein refers to a composition or kit of parts where the combination partners as defined above can be dosed dependently or independently or by use of different fixed combinations with distinguished amounts of the combination partners, i.e., simultaneously or at different time points. The combination partners can then, e.g., be administered simultaneously or chronologically staggered, that is at different time points and with equal or different time intervals for any part of the kit of parts. The ratio of the total amounts of the combination partners to be administered in the combination can be varied, e.g. in order to cope with the needs of a patient sub-population to be treated or the needs of the single patient which different needs can be due to age, sex, body weight, etc. of the patients.

It is especially advantageous to formulate the lipase inhibitors and pharmaceutical formulations in dosage unit form for ease of administration and uniformity of dosage.

Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the novel dosage unit forms of the invention are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired as herein disclosed in detail.

As mentioned above the principal active ingredient may be compounded for convenient and effective administration in therapeutically effective amounts with a suitable pharmaceutically acceptable vehicle in dosage unit form. A unit dosage form can, for example, contain the lipase inhibitor in amounts ranging from 0.25 μg to about 2000 mg. Expressed in proportions, the active compound may be present in from about 0.25 μg to about 2000 mg/mL of carrier. In the case of compositions containing supplementary active ingredients, the dosages are determined by reference to the usual dose and manner of administration of the said ingredients.

As used herein, the term “effective amount” refers to an amount of compound which, when administered according to a desired dosing regimen, provides the desired therapeutic activity. Dosing may occur once, or at intervals of minutes or hours, or continuously over any one of these periods. Suitable dosages may lie within the range of about 0.1 ng per kg of body weight to 1 g per kg of body weight per dosage. A typical dosage is in the range of 1 μg to 1 g per kg of body weight per dosage, such as is in the range of 1 mg to 1 g per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 100 mg per kg of body weight per dosage, such as up to 50 mg per kg of body weight per dosage.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

The reference in this specification to any prior publication (or information derived from it), or to any matter which is known, is not, and should not be taken as an acknowledgment or admission or any form of suggestion that prior publication (or information derived from it) or known matter forms part of the common general knowledge in the field of endeavour to which this specification relates.

The invention will now be described with reference to the following non-limiting examples. The following examples are representative of the general formula (I) and provide detailed methods for preparing exemplary compounds of the present invention.

### Example 1. Intestinal Delivery in a Long-Chain Fatty Acid Formulation Enables Mesenteric Lymphatic Transport and Systemic Exposure of Orlistat

### 1.1 Methods

### 1.1.1 Experimental Design

A range of lipid-based formulations, as detailed in Table 1, were prepared to evaluate the effect of lipid type and dose on the intestinal absorption, lymphatic transport and systemic availability of orlistat. Lymphatic transport was determined in a triple cannulated anaesthetised rat model with cannulas inserted into the mesenteric lymph duct, carotid artery and duodenum for lymph and blood collection, and formulation infusion, respectively. Systemic availability was determined in lymph-intact anaesthetised rats with cannulas inserted into the carotid artery and duodenum for blood collection and formulation infusion, respectively. To enable the calculation of absolute bioavailability an additional group was administered orlistat intravenously via a jugular vein cannula, and blood samples collected from a carotid artery cannula over time.

TABLE 1
Experimental groups including formulations, studies conducted,
and administration details. All formulations were prepared
in 5.6 ml PBS and infused intraduodenally at a rate of 2.8
ml/h for 2 h unless specified otherwise. Lymphatic uptake
studies were conducted for all experimental groups except
for the intravenously administered group in the last row.

| | | |
|---|---|---|
| Orlistat | | Tween 80 |
| dose | Lipid type & dose | dose |

| | | | |
|---|---|---|---|
| 8 | mg/kg | No lipid control | 112 mg |
| 8 | mg/kg | 40 mg Octanoic Acid | 25 mg |
| 8 | mg/kg^{a} | 40 mg Olive Oil | 25 mg |
| 8 | mg/kg^{a} | 40 mg Oleic Acid | 25 mg |
| 8 | mg/kg | 80 mg Oleic Acid | 25 mg |

^{a}Bioavailability studies were conducted

To determine the effect of lipid type on orlistat absorption, three different types of lipids were tested: a long chain fatty acid in the form of oleic acid (LC-FA), a medium chain fatty acid (octanoic acid, MC-FA), and a long chain triglyceride (olive oil, LC-TG). In addition, a lipid free formulation was evaluated as a control formulation. After determining that co-administration with oleic acid (LC-FA) promoted the highest absorption and lymphatic transport of orlistat, further investigations focused on oleic acid based formulations. Three lipid doses were tested: 0 mg oleic acid, 40 mg oleic acid and 80 mg oleic acid. Drug dose was kept consistent at 8 mg/kg.

### 1.1.2 Preparation of Orlistat Lipid-Based and Control Formulations.

### 1.1.2.1 Orlistat Lipid-Based Formulations for Intestinal Administration

Various lipid-based formulations of orlistat were prepared as listed in Table 2. Orlistat was added into a glass vial, mixed with the lipids and Tween 80 at the required concentration for each formulation as per Table 1 and was incubated at 37° C. for 2 h followed by 10-12 h incubation at room temperature. Subsequently, the required volume of phosphate buffered saline (PBS, pH 7.4) was added to the lipid phase (i.e. the orlistat, lipid and Tween 80 mixture). The formulations were emulsified with a Misonix XL 2020 ultrasonic processor (Misonix, Farmingdale, NY, USA) or a Qsonica Q700 ultrasonic processor (Qsonica, CT, USA). Drug concentration in the formulations was confirmed by LC-MS/MS analysis as below.

### 1.1.2.2 Orlistat Lipid Free Formulation for Intestinal Administration

For the lipid-free control formulations orlistat was dissolved in Tween 80 at the required concentrations as defined in Table 2. Subsequently, the required volume of PBS (pH 7.4) was added. Then, the formulation was emulsified with a Soniclean 160HT ultrasonic cleaner for 30 minutes at room temperature. Drug concentration in the formulations was confirmed by LC-MS/MS analysis as below.

### 1.1.2.3 Orlistat Formulation for Intravenous Infusion

To prepare the intravenous orlistat formulation, 0.1 mg of orlistat was dissolved in 120 mg soybean oil in a glass vial. Subsequently, 1 ml of 2% glycerol and 1% EPC in water was added to the lipid phase and the formulation was emulsified with the same ultrasonic processor, microprobe tip and ultrasonication settings as described for the lipid-based formulations for 1 h at intervals of 5 min on and 5 sec off, in an ice water bath.

### 1.1.3 Animal Studies

All animal experiments were approved by the local animal ethics committee and were conducted in accordance with the Australian and New Zealand Council for the Care of Animals in Research and Teaching guidelines. Male Wistar rats ranging from 260-310 g were maintained on a standard diet and fasted overnight (14-16 h) with free access to water prior to commencement of the experiment. Anaesthesia was induced and maintained with 1.5-5% v/v isoflurane, delivered via a nose cone. To maintain body temperature rats were kept on a 37° C. heating pad throughout the experiments.

### 1.1.3.1 Lymphatic Transport Studies

The lymphatic transport and plasma concentrations of orlistat, were assessed after administration of all formulations. In these experiments, the mesenteric lymph duct, carotid artery and duodenum were cannulated as described previously (Trevaskis, N. L., et al., J Vis. Exp. 2015, 6(97), p. e52389; Edwards, G. A., et al., Advanced Drug Delivery Reviews, 2001, 50(1), 45-60). After surgery, the rats were hydrated for at least 30 min via intraduodenal infusion of normal saline at 2.8 ml/h. The control or lipid-based orlistat formulations were then infused into the duodenum at 2.8 ml/h for 2 h. Following completion of formulation dosing, the intraduodenal infusion was switched back to 2.8 ml/h normal saline for the remainder of the experiment. Mesenteric lymph was collected continuously for 8 h following commencement of formulation dosing, into pre-weighed polyethylene tubes containing 10-20 μl of 1000 IU/ml heparin. Lymph collection tubes were changed every hour and lymph flow was determined gravimetrically. Aliquots of 100 μl of lymph were stored at −20° C. for later drug or TG analysis by HPLC-MS/MS. 250 μl of blood was also collected from the carotid artery and placed into polyethylene tubes together with 3 μl of 1000 IU/ml heparin at 10 time points: 0 min, 5 min, 15 min, 30 min, 1 h, 2 h, 3 h, 4 h, 6 h, and 8 h. Blood samples were centrifuged for 5 min at 2000 μg to separate plasma.

### 1.1.3.2 Plasma Pharmacokinetic and Bioavailability Studies

To enable determination of systemic availability following intestinal dosing of selected formulations (i.e. the 40 mg oleic acid and olive oil formulations containing 8 mg/kg orlistat) a separate group of lymph-intact animals (i.e. non-lymph cannulated) were cannulated at the duodenum and carotid artery to enable formulation administration and blood collection, respectively. After surgery the rats were hydrated via intraduodenal infusion of 2.8 ml/h normal saline for at least 30 min, followed by intraduodenal infusion of the formulations at 2.8 ml/h for 2 h. The hydration of the rats, collection of blood samples, separation of plasma, and euthanasia were the same as described for the lymphatic transport studies.

An intravenous pharmacokinetic study was also completed to enable the calculation of the absolute bioavailability following intestinal administration. In this, the jugular vein and carotid artery of the rats were cannulated as described above. After surgery, the rats were rehydrated via the jugular vein infusion of normal saline for at least 30 min at 0.5 ml/h. An orlistat (0.4 mg/kg) lipid emulsion (2% glycerol, 1% egg phosphatidylcholine in PBS) was administered as a short 1 ml IV infusion into the jugular vein cannula over 5 min. Following completion of formulation dosing, the jugular vein infusion was switched back to 0.5 ml/h normal saline for the remainder of the experiment. The collection of blood samples, separation of plasma, and euthanasia were the same as described for the lymphatic transport studies.

### 1.1.4 HPLC-MS/MS Sample Preparation and Analysis

Lymph and plasma concentrations of orlistat in open ring form (less active at inhibiting PLs) and closed ring form (most active at inhibiting PLs) were quantified by HPLC-MS/MS. Concentrations of orlistat in lymph, plasma and formulations were analysed using a Shimadzu LCMS-8050 system (Shimadzu Scientific Instruments, Kyoto, Japan) consisting of a CBM-20A system controller, a DGU-20A5R degassing unit, two Nexera X2 LC-30 AD liquid chromatograph pumps, a Nexera X2 SIL-30AC autosampler, a CTO-20A column oven (held at 40° C.), and a LCMS-8050 triple quadrupole mass spectrometer with an atmospheric-pressure chemical ionization (APCI) interface.

### 1.1.5 Data Analysis for Lymphatic Transport, Pharmacokinetic and Bioavailability Studies

For the lymphatic transport studies, mass transport of orlistat in lymph was calculated by multiplying the volume of lymph collected by the measured concentration of orlistat in lymph determined by HPLC-MS/MS analysis. Lymph:plasma concentration ratios were calculated by dividing the average orlistat lymph concentration for each hourly collection period by the orlistat plasma concentration measured at the end of the hourly collection period.

The plasma concentration was set at the lower limit of quantification (i.e. 0.05 μg/ml) for timepoints where the plasma concentration was below 0.05 μg/ml when determining the lymph:plasma concentration ratio.

For the plasma pharmacokinetic studies, the absolute bioavailability following intestinal delivery of orlistat was calculated from:

$$Absolute bioavailability = \frac{{AUC}_{0 - 8 h} intestinally administered}{{AUC}_{0 - 8 h} IV administered} \times \frac{Orlistat {Dose}_{IV}}{Orlistat {Dose}_{intestinal}}$$

Where AUC₀₋₈ₕ intestinally administered and IV administered are the area under the plasma concentration time curves from time 0 to 8 h after intestinal and IV administration, respectively. Plasma AUCs were calculated using the linear trapezoidal method.

For the IV administered group, non-compartmental and compartmental pharmacokinetic parameters (normalised to 0.4 mg/kg orlistat) were calculated using WinNolin® Software (WinNolin® professional version 5.2.1, Pharsight Corporation, CA, USA).

### 1.1.6 Statistics

GraphPad Prism for Windows V7.01.180 (GraphPad Software Inc. Ca, USA) was used to perform statistical analyses. One-way ANOVA followed by Tukey's multiple comparisons test (for comparisons between three or more groups) or an unpaired t test (for comparisons between two groups) was used to determine significant differences with a level of p=0.05 set as significant (unless otherwise noted).

### 1.2 Results

### 1.2.1 the Effect of Lipid Type on the Lymphatic Transport of Orlistat and Triglyceride

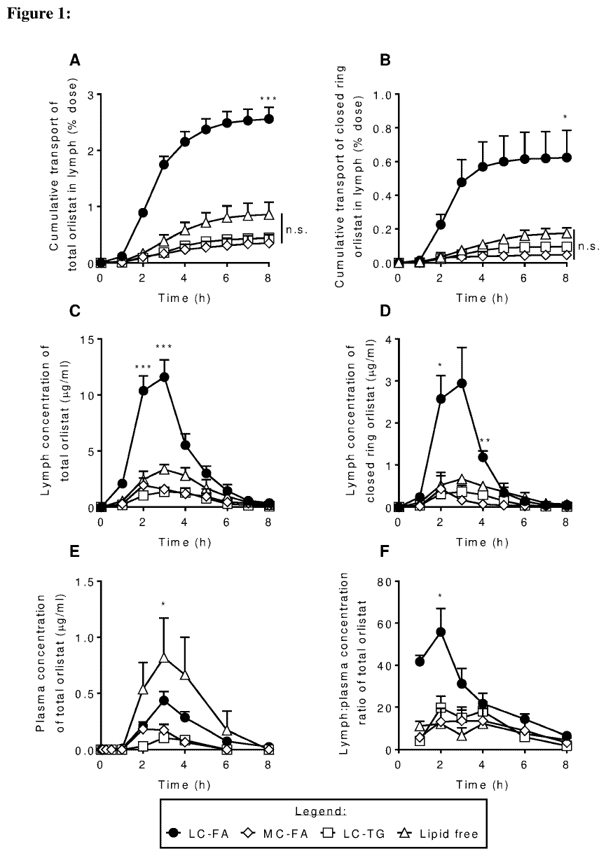

The cumulative lymphatic transport of orlistat was significantly greater when it was administered in the LC-FA (oleic acid) based formulation (at 2.6% of the dose for total orlistat and 0.6% of dose for closed-ring orlistat over 8 h) when compared to the lipid free (i.e. control), LC-TG (olive oil) and MC-FA (octanoic acid) based formulations for which lymphatic transport was relatively low at <0.9% of dose over 8 h for total orlistat (FIG. 1, Panel A, and Panel B and Table 2). The LC-TG and MC-FA based formulations therefore did not promote lymphatic transport of orlistat relative to the lipid-free formulation.

The peak concentration (Cₘₐₓ) of orlistat in lymph generally occurred at 2-3 h post-dose for all formulations after which time the lymph concentrations of orlistat declined. The orlistat Cₘₐₓ in lymph was significantly higher when it was administered in the LC-FA formulation when compared to the LC-TG, MC-FA and lipid free formulations, as was expected from the higher cumulative lymphatic transport (FIG. 1, Panel A-D).

TABLE 2
Summary of mesenteric lymphatic transport and plasma Cmax for orlistat
in lymph cannulated rats administered the different formulations.

| | | Cumulative % | |
|---|---|---|---|
| | Cumulative % | closed form orlistat | Lymph Cmax of |
| | total orlistat dose | dose in lymph over | total orlistat |
| Formulation dosed | in lymph over 8 h | 8 h | (μg/ml) |
| 8 mg/kg orlistat in | 0.86 ± 0.38% | 0.17 ± 0.03% | 3.36 ± 0.43 |
| no lipid (control) | | | |
| 8 mg/kg orlistat in | 0.33 ± 0.15% | 0.05 ± 0.03% | 1.60 ± 0.39 |
| 40 mg MC-FA | | | |
| 8 mg/kg orlistat in | 0.44 ± 0.06% | 0.10 ± 0.02% | 1.36 ± 0.36 |
| 40 mg LC-TG | | | |
| 8 mg/kg orlistat in | 2.56 ± 0.21% ^{a} | 0.62 ± 0.16% | 10.9 ± 1.09 |
| 40 mg LC-FA | | | |
| 8 mg/kg orlistat in | 3.62 ± 0.23% | 1.57 ± 0.11% ^{b} | 21.13 ± 0.93 |
| 80 mg LC-FA | | | |

| | Lymph Cmax of | | Cumulative TG |
|---|---|---|---|
| | closed form | Plasma Cmax of | mass transport |
| | orlistat | total orlistat | in lymph (mg) |
| Formulation dosed | (μg/ml) | (μg/ml) | over 8 h |
| 8 mg/kg orlistat in | 0.67 ± 0.09 | 0.82 ± 0.35 | 26.36 ± 2.67 |
| no lipid (control) | | | |
| 8 mg/kg orlistat in | 0.16 ± 0.07 | 0.17 ± 0.05 | 15.78 ± 4.49 |
| 40 mg MC-FA | | | |
| 8 mg/kg orlistat in | 0.37 ± 0.16 | 0.10 ± 0.02 | 22.16 ± 2.84 |
| 40 mg LC-TG | | | |
| 8 mg/kg orlistat in | 2.94 ± 0.85 | 0.44 ± 0.08 | 35.7 ± 0.64 |
| 40 mg LC-FA | | | |
| 8 mg/kg orlistat in | 9.08 ± 1.38 | 0.31 ± 0.07 | 31.77 ± 4.22 |
| 80 mg LC-FA | | | |

n = 3-6 rats per group, data is presented as mean ± SEM. Statistical significance between groups was determined by one-way ANOVA.
^{a} Significantly greater when compared to all other experimental groups for the same parameter except for the groups administered 8 mg/kg orlistat in 80 mg LC-FA (2 h infusion) and 30 mg/kg orlistat in 160 mg LC-FA (8 h infusion) (p ≤ 0.001)
^{b} Significantly greater when compared to all other experimental groups for the same parameter except for the group administered 32 mg/kg orlistat in 160 mg LC-FA (8 h infusion) (p ≤ 0.001)

In the plasma of lymph cannulated animals, orlistat was only measurable in open ring form. The closed ring form was present in some samples but below the limit of quantitation. In contrast to the lymph profiles, orlistat plasma concentrations were substantially higher after administration in the lipid free formulation when compared to the lipid-based formulations (FIG. 1, Panel E). Across all formulations, plasma Cₘₐₓ generally occurred between 2-3 h post-dosing followed by a decline beyond 6 h post-dosing. The plasma concentrations of orlistat were lower (2-56 fold) than in lymph across all time points and in all groups. The lymph:plasma concentration ratio of total orlistat at 3 h post-dose was significantly higher following administration of the LC-FA formulation when compared to the other formulations (FIG. 1, Panel F).

### 1.22 the Effect of Lipid Dose on the Lymphatic Transport of Orlistat

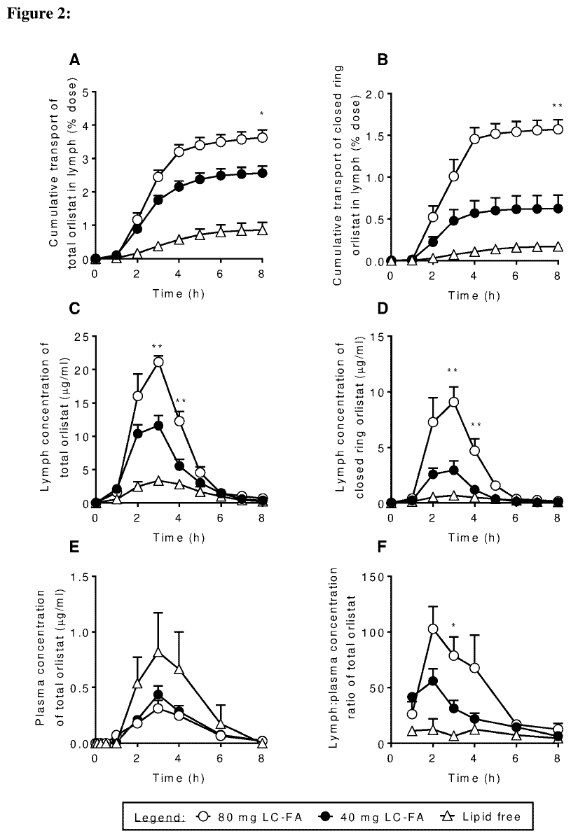

The LC-FA based formulation therefore supported increased lymphatic transport of orlistat compared to the other LBFs and lipid free formulation (FIG. 1). Next, the impact of increasing the LC-FA (i.e. oleic acid) dose from 40 mg to 80 mg while keeping the drug dose constant was tested. Doubling the LC-FA dose to 80 mg significantly enhanced the lymphatic transport of orlistat. The mean transport of total orlistat (open and closed ring forms) in lymph was 2.6% and 3.6% of the dose, and the mean transport of the more active closed ring form of orlistat was 0.6% and 1.6% of the dose, for the 40 mg and 80 mg LC-FA formulations, respectively. Both LC-FA formulations enhanced orlistat lymphatic transport when compared to the lipid-free control (FIG. 2, Panel A and B, and Table 3). The lymph Cₘₐₓ for total orlistat and closed ring form of orlistat (i.e. 2-3 h time point) was also significantly higher after co-administration with 80 mg when compared to 40 mg of LC-FA (FIG. 2, Panel C and D). Both LC-FA formulations resulted in higher concentrations of orlistat in lymph when compared to plasma (FIG. 2, Panel E). In contrast to the differences seen in lymph concentrations, the plasma concentrations of total orlistat (which was almost entirely present in open ring form in plasma) were similar after co-administration with either 40 mg or 80 mg of oleic acid (FIG. 2, Panel E). The lymph to plasma concentration ratio of total orlistat was thus greater following administration with 80 mg when compared to 40 mg of LC-FA for up to 4 h post dose (FIG. 2, Panel F).

### 1.23 the Bioavailability and Systemic Exposure of Orlistat

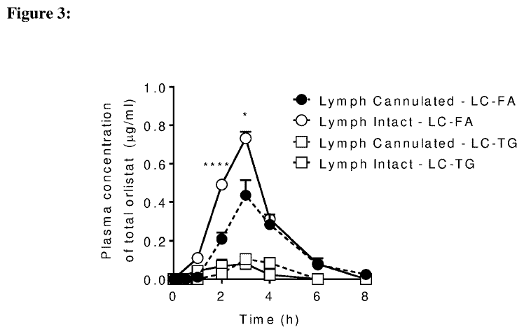

To further quantify the contribution of lymphatic transport to the systemic availability of orlistat after administration in the LBFs, the area under the plasma concentration versus time profiles (AUC) of total orlistat were compared in lymph intact and lymph cannulated rats administered the LC-FA and LC-TG based formulations. The absolute bioavailability of total orlistat in the groups administered these formulations was also determined by comparing the plasma AUC of total orlistat to rats administered orlistat 0.4 mg/kg IV. In both the groups administered orlistat intestinally and IV, plasma orlistat was almost entirely present in the open ring form suggesting that orlistat was rapidly hydrolysed in the systemic circulation. The closed ring form was detected in some samples but was below the limit of quantitation of the assay (0.05 μg/ml). Plasma concentrations over time and the bioavailability attributed by the orlistat open ring form were substantially higher for the LC-FA (bioavailability of 3.9%) when compared to the LC-TG formulation (bioavailability of 0.5%) (FIG. 3 and Table 3). In all rats administered orlistat into the intestine, the plasma Cₘₐₓ of orlistat occurred at 3-4 h post-dose followed by a decline beyond 6 h. In the rats administered the LC-TG based formulation there was no significant difference in total orlistat plasma concentrations between the lymph intact and lymph cannulated groups. In contrast, after dosing the LC-FA formulation the orlistat plasma concentrations were significantly greater in lymph intact versus lymph cannulated rats at 2 and 3 h after commencing dosing (FIG. 3). By determining the proportional reduction in the plasma AUC in lymph intact versus lymph cannulated/diverted rats, ˜37 and 0 percent of systemic exposure of orlistat was estimated to be contributed by the lymphatically transported drug for the LC-FA and LC-TG formulations, respectively. Therefore, a substantially greater percentage of systemic exposure appeared to be contributed by lymphatic transport for the LC-FA based formulation when compared to the LC-TG formulation. This is consistent with the orlistat lymphatic transport data (Table 3).

TABLE 3
Summary of the orlistat plasma and lymph pharmacokinetic data in lymph cannulated
(LC) vs lymph intact (LI) rats administered 8 mg/kg orlistat in 40 mg LC-FA
or LC-TG. Data is presented as mean ± SEM for n = 3-5 rats. Note
that orlistat in plasma was almost entirely in open ring form in all animals.

| Experiment group | LC-FA - LC | LC-FA - LI | LC-TG -LC | LC-TG - LI |
|---|---|---|---|---|
| Plasma Cₘₐₓ | 0.44 ± 0.08 | 0.73 ± 0.03 | 0.10 ± 0.01 | 0.24 ± 0.25 |
| (μg/ml) | | | | |
| Plasma AUC | 1.33 ± 0.34 | 1.99 ± 0.06 | 0.33 ± 0.13 | 0.38 ± 0.01 |
| (μg*h/ml) | | | | |
| Absolute | 2.41 ± 0.61% | 3.60 ± 0.11% | 0.59 ± 0.23% | 0.69 ± 0.02% |
| bioavailability (%) | | | | |
| Lymphatic | 2.56 ± 0.21% | 1.49% ^{f} | 0.44 ± 0.06% | 0.00% |
| transport (% dose | | | | |
| over 8 h) | | | | |

### Example 2. Intestinal Delivery of the Pancreatic Lipase Inhibitor Orlistat Mitigates Lymph Cytotoxicity and Disease Severity in Acute Pancreatitis

### 2.1 Methods

### 21.1 Experimental Design

To determine the potential to mitigate acute pancreatitis disease severity via inhibition of pancreatic lipases (PLs) in the gut-lymph and/or gastrointestinal lumen, a total of six groups (three lymph diverted and three lymph intact), were conducted as per Table 4.

In the previous study it was determined in healthy rats that orlistat administration in a long chain fatty acid (LC-FA) formulation promotes lymphatic drug transport when compared to administration in a lipid free formulation (LFF). However, the extent of lymphatic transport and systemic exposure may differ in acute pancreatitis because of enhanced gut permeability due to gut ischemia (P. O. Juvonen, E. M. A. J. A. T., Scandinavian Journal of Gastroenterology, 2000, 35(12), 1314-1318; Wu, X.-N., World Journal of Gastroenterology, 2000, 6(1), 32-36). Therefore, blank, LC-FA formulation, orlistat LC-FA formulation and LFF were dosed to AP rats and sham rats that were lymph diverted externally or lymph intact. I n the lymph diverted rats, lymph and matching blood samples were collected to enable measurements of orlistat concentration in lymph and plasma, SIRS/MODS marker concentration in serum, and gut-lymph cytotoxicity. In the lymph intact rats, blood samples were collected to analyse orlistat plasma concentrations and markers of SIRS. Blood pressure and heart rate were also determined.

TABLE 4
Experimental groups including lymph intervention,
animal model and formulation administered

| Lymph | | | |
|---|---|---|---|
| intervention | Animal Model | Formulation | Drug |
| Lymph diverted | Sham | LC-FA | orlistat |
| Lymph diverted | Acute Pancreatitis | LC-FA | NIL (Blank) |
| Lymph diverted | Acute Pancreatitis | LC-FA | orlistat |
| Lymph intact | Acute Pancreatitis | LC-FA | NIL (Blank) |
| Lymph intact | Acute Pancreatitis | LC-FA | orlistat |
| Lymph intact | Acute Pancreatitis | LFF | orlistat |

### 21.2 Preparation of Lipid-Based Formulation and Control Formulation

Preparation of the orlistat LC-FA formulation and LFF is as described above.

### 21.3 Animals

All animal experiments were approved by the University of Auckland Animal Ethics Committee (approval number R1985). Male Sprague-Dawley rats ranging 420-470 g were maintained on a standard 18% protein rodent diet and fasted overnight (14-16 h) with free access to water prior to commencement of the experiment. General anaesthesia was induced in an induction chamber with 5% v/v isoflurane and oxygen at 2 L/min and maintained with 1.5-3% v/v isoflurane using a nose cone. Subcutaneous injection of buprenorphine hydrochloride (30 μg/kg body weight; Temgesic, SVS) was given for pain relief. To ensure body temperature was maintained, rats were kept on a 37° C. heating pad throughout the experiments, and the temperature was constantly monitored via a rectal thermometer probe (ADinstruments).

### 2.1.3.1 Acute Pancreatitis Model

The acute pancreatitis (AP) model was a variation of an established model to allow blood collection at different time points and the continuous collection of lymph (Mittal, A., et al., JOP, 2009, 10(2), 130-42; Shanbhag, S. T., et al., Surgery, 2018, 163(5), 1097-1105). In this model, a tracheostomy (for respiration), insertion of a pressure transducer into the femoral artery (2F Mikro-Tip® rat pressure transducer Cat #SPR-320; Millar Instruments Inc., USA, for monitoring of vitals) and cannulation of the carotid artery (for blood sampling), femoral vein (for resuscitation), duodenum (for formulation dosing), mesenteric lymph duct (for lymph collection), and biliopancreatic duct (for AP induction) were conducted.

After surgery, the orlistat/blank LC-FA formulations and orlistat LFF were then infused into the duodenum at 2.8 ml/h until the end of the experiment (i.e. 4 h post initiation of the infusion). For groups with AP, 0.5 h after initiation of the orlistat formulation infusion, sodium tauorocholate (5% w/v in saline) was infused into the biliopancreatic duct to induce AP.

### 2.1.3.2 Lymph and Blood Sampling

After initiation of dosing of the formulations, lymph was collected continuously for the duration of the experiment (i.e. up to 4 h post initiation of formulation dosing) into pre-weighed 1.5 ml tubes that were kept in an ice bath. Lymph collection tubes were changed every hour and lymph flow was determined gravimetrically. Aliquots of 100 μl of lymph were placed in 1.5 ml Eppendorf tubes with 1 μl of 1000 IU/ml heparin for analysis of orlistat by HPLC-MS/MS as described above. Aliquots of 100 μl of lymph were placed in 1.5 ml Eppendorf tubes without heparin for in vitro lymph cytotoxicity assessment. All samples were kept at −80° C. for long term storage. 250 μl of blood from the carotid artery was also collected into Eppendorf tubes together with 3 μl of 1000 IU/ml heparin at 5 time points: 0 h, 12 h, 3 h, and 4 h post formulation dosing, to determine plasma orlistat concentration. Blood samples were centrifuged for 5 min at 2000×g to separate plasma. Plasma was stored as described for the lymph samples. A terminal blood collection was conducted via the carotid artery cannula. Blood was immediately transferred to BD plastic vacutainer tubes to collect serum for biochemical, cytokine and cardiac injury marker analysis. Serum and plasma samples from the terminal blood were kept at −80° C. for long term storage. Cytokine and cardiac injury marker analysis was conducted using serum samples with Milliplex rat cytokine/chemokine and rat cardiac injury magnetic bead panel 96-well plate assay kits as per manufacturer protocol (Merck, Germany).

### 21.4 Lymph Cytotoxicity Assay

Rat lung epithelial cells (L2, ATCC CCL-149) and human dermal microvascular endothelial cells (HMEC-1, ATCC CRL-3243) were utilized to determine the toxicity of gut-lymph. For L2 cells, the complete growth medium was 10% FBS and 100 U/ml penicillin-streptomycin in Ham's F-12K (Kaighn's) medium. The complete growth medium for HMEC-1 cells was 10% FBS, 10 mM L-glutamine, 1 μg/ml hydrocortisone, and 10 ng/ml human recombinant epidermal growth factor in MCDB 131 medium. Both cell lines were cultured in their respective complete growth media in 75 cm² tissue culture flasks to confluency at 37° C. in 95% air/5% CO₂. The cells were seeded at 10,000 cells/well for HMEC-1 and 5,000 cells/well for L2 in CellCarrier-96 well black plates with optically clear bottom (PerkinElmer, MA, USA) in 95 μl/well growth medium without FBS. Then 5 μl (equivalent 5% v/v) of gut-lymph sample was added per well in duplicate. Only lymph that was collected 0.5 h and 2.5 h post AP induction was tested for lymph cytotoxicity due to limited lymph sample volume collected from some rats. 5 μl/well (equivalent to 5% v/v) of FBS, PBS or no addition were added in other wells on the same plate in quadruplicate as controls. Cells were incubated for 24 h at 37° C. in 95% air/5% CO₂ with the treatments. After 24 h incubation, 2× detection reagent was prepared from CyQUANT™ Direct Cell Proliferation Assay Kit (Invitrogen, CA, USA) as per manufacturer's protocol, and 100 μl of 2× detection reagent was added to each well and incubated for 1 h at 37° C. in 95% air/5% CO₂. The CyQUANT™ kit measures cell viability via a cell-permeant DNA-binding dye in combination with a masking dye; the masking dye blocks staining of cells with compromised cell membranes (i.e. dead cells) and thus only healthy cells are stained. ‘No cell controls’ were prepared by mixing 100 μl of test medium and 100 μl of 2× detection reagent, to determine background fluorescence. Microscopic images were captured using the Operetta High Content Imaging System (PerkinElmer, MA, USA) at 4 different locations each well using ×20 long WD objective lens under brightfield and FITC channels. Total fluorescence was determined using the iD3 SpectraMax plate reader (Molecular Devices, CA, USA) at ex/em 480/535 nm under bottom-read setting.

### 21.5 Serum Biochemical Composition

Analyte concentrations were measured in sera on the Cobas c311 clinical chemistry analyser (Roche, Mannhelm, Germany) with the following methods: enzymatic colorimetric (glucose, lipase, TG, cholesterol); enzyme-linked kinetic ultraviolet [alanine transaminase (ALT), aspartate aminotransferase (AST)]; kinetic ultraviolet (urea); kinetic colorimetric (creatinine); colorimetric (total protein, albumin, ALP); UV-Test (creatine kinase); direct potentiometric using ion selective electrodes (calcium, sodium, potassium, chloride). All the reagents required for the protocols were purchased from Roche (Roche, Mannheim, Germany).

### 21.6 Serum Cytokine Assay

To screen for several cytokines at once, and reduce sample wastage, Milliplex rat cytokine/chemokine magnetic bead panel 96-well plate assay kits were utilised. These kits enable the ability to acquire the levels of multiple cytokines per sample via Luminex® xMAP® technology which involves internally colour-coded microspheres coupling with analyte of interest followed by incubation with a reporter dye.

### 21.7 Data Analysis

### 2.1.7.1 Lymphatic Transport and Pharmacokinetic

Calculations were as described above.

### 2.1.7.2 Lymph Cytotoxicity Study

Background fluorescence was subtracted from all the wells and % viability after lymph treatment was determined by dividing the corrected fluorescence of each treatment group relative to the average corrected fluorescence of control media (5% FBS) and multiplying the ratio to 100. D'Agostino & Pearson normality test and Shapiro-Wilk normality test were used to check the data normality.

### 2.1.8 Statistics

GraphPad Prism for Windows V7.01.180 (GraphPad Software Inc. Ca, USA) was used to perform statistical analyses. One-way ANOVA followed by Tukey's multiple comparisons test (for comparisons between 3 or more groups) or an unpaired t test (for comparisons between 2 groups) was used to determine significant differences with a level of p=0.05 set as significant.

### 2.2 Results

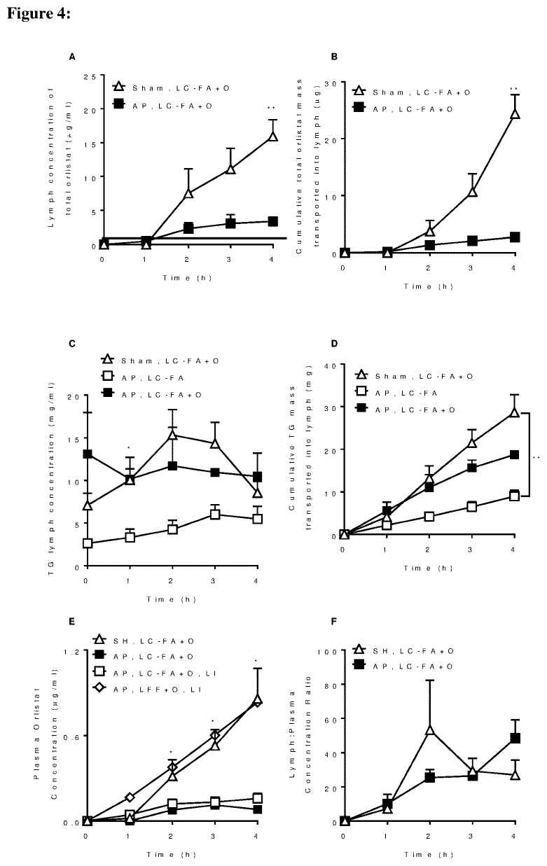

In animals with AP it was found that the gut-lymph uptake of orlistat was attenuated relative to healthy animals due to reduced lymph flow and reduced lipid/drug absorption associated with the disease (FIG. 4). However, the LC-FA formulation was still able to deliver orlistat into gut-lymph at therapeutic concentrations in the rats with AP.

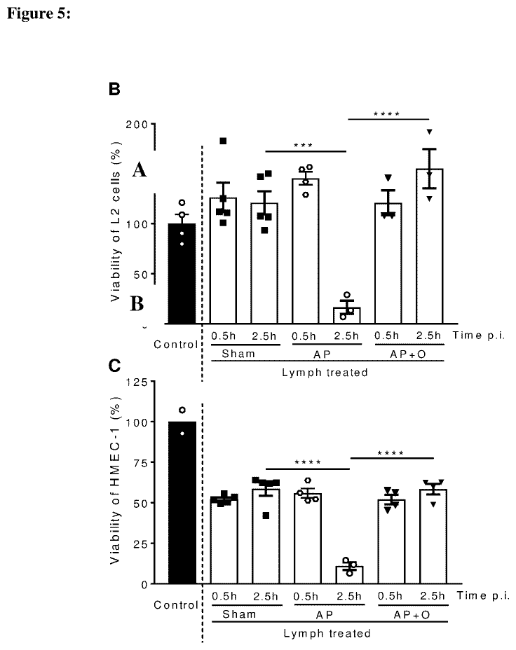

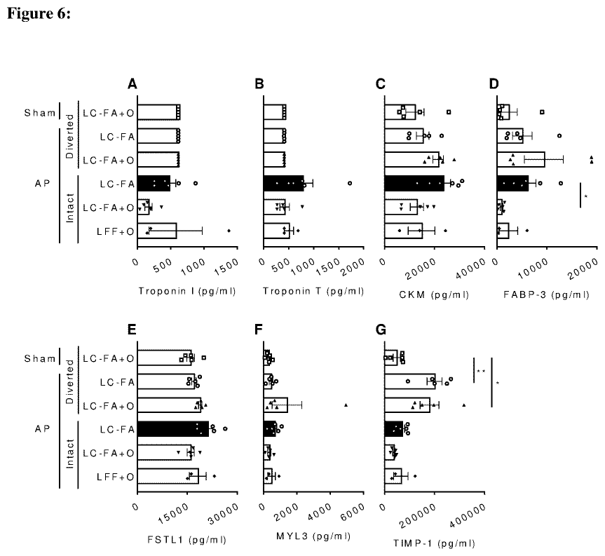

Administration of orlistat LC-FA to AP rats reduced lymph cytotoxicity to L2 lung and HMEC-1 vascular endothelial cells (FIG. 5). In addition, AP rats dosed with orlistat LC-FA had reduced serum cardiac injury marker levels (FIG. 6), changes to serum biochemistry (Table 5) and improved blood pressure (BP) (Table 6) when compared to rats dosed with blank LC-FA formulation. AP rats administered orlistat LFF appeared to also show some reduction in cardiac dysfunction; however, there was a trend toward improved efficacy for the lymph directing LC-FA formulation. Overall, enteral administration of the orlistat LC-FA formulation to AP rats enhanced orlistat uptake into lymph, reduced lymph cytotoxicity and cardiac injury. These findings support that enteral administration of orlistat in our lymph-directing LC-FA formulation can provide an effective and specific treatment for lipase induced injury from severe AP.

TABLE 5
Serum biomarkers of disease severity in the different experimental groups.

| | Lymph diverted | | |
|---|---|---|---|
| | Sham | Acute pancreatitis | |
| | LC-FA + O | LC-FA | LC-FA + O |
| Na⁺ (mmol/L) | 145.2 ± 0.7 | 145.2 ± 0.9 | 142.8 ± 0.4 |
| K⁺ (mmol/L) | 5.8 ± 0.1 | 7.1 ± 0.2^{a} | 14.8 ± 4.9^{a} |
| Cl⁻ (mmol/L) | 106.3 ± 0.3 | 106.0 ± 0.5^{a} | 103.2 ± 1.6^{a} |
| Ca²⁺ (mmol/L) | 2.2 ± 0.0 | 2.1 ± 0.0 | 2.2^{d} |
| TP (g/L) | 45.9 ± 0.9 | 48.1 ± 0.9 | 48.1 ± 0.4 |
| Albumin (g/L) | 29.3 ± 1.0 | 30.3 ± 0.9 | 31.2 ± 0.1 |
| CK (U/L) | 1610.0 ± 318.0 | 2117.8 ± 411.3 | 3620.0 ± 1256.2 |
| ALT (U/L) | 66.4 ± 7.8 | 115.4 ± 11.2^{a} | 173.4 ± 29.0^{a} |
| AST (U/L) | 230.4 ± 11.0 | 455.5 ± 73.1^{a} | 699.6 ± 171.3^{a} |
| ALP (U/L) | 145.8 ± 15.0 | 148.2 ± 15.8 | 108.2 ± 41.2 |
| Creatine (μmol/L) | 43.6 ± 5.0 | 119.4 ± 5.0^{a} | 118.4 ± 9.7^{a} |
| Urea (mmol/L) | 14.7 ± 1.4 | 19.7 ± 0.7^{a} | 18.2 ± 0.5 |
| Pancreatic lipase | 11.9 ± 2.5 | 346.5 ± 51.9^{a} | 481.0 ± 182.0^{a} |
| (U/L) | | | |
| TG (mmol/L) | 1.4 ± 0.3 | 0.4 ± 0.0^{a} | 0.5 ± 0.1^{a} |
| Chol (mmol/L) | 1.4 ± 0.1 | 1.9 ± 0.1 | 1.6 ± 0.1 |
| Glucose (mmol/L) | 10.8 ± 0.6 | 11.5 ± 0.7 | 10.5 ± 1.1 |

| | Lymph intact - Acute pancreatitis | | |
|---|---|---|---|
| | LC-FA | LC-FA + O | LFF + O |
| Na⁺ (mmol/L) | 146.2 ± 0.6^{b} | 147.0 ± 0.7^{b} | 150 ± 86.6 |
| K⁺ (mmol/L) | 7.5 ± 0.3 | 6.5 ± 0.2 | 6.8 ± 3.9 |
| Cl⁻ (mmol/L) | 108.7 ± 0.6 | 110.9 ± 0.6 | 110.6 ± 63.8 |
| Ca²⁺ (mmol/L) | 2.1 ± 0.1 | 2.4 ± 0.0 | 2.1 ± 1.2 |
| TP (g/L) | 49.0 ± 1.2 | 46.9 ± 1.5 | 44.8 ± 25.8 |
| Albumin (g/L) | 33.4 ± 1.3 | 30.1 ± 1.4 | 28.6 ± 16.5 |
| CK (U/L) | 4085.8 ± 808.7 | 1775.8 ± 231.1^{c} | 2634.3 ± 1520.9 |
| ALT (U/L) | 359.6 ± 144.0 | 163.4 ± 67.8 | 124.6 ± 71.9 |
| AST (U/L) | 1763.7 ± 928.8 | 524.3 ± 145.7 | 609.5 ± 351.9 |
| ALP (U/L) | 180.5 ± 9.7 | 159.8 ± 17.1 | 141.0 ± 81.4 |
| Creatine (μmol/L) | 124.0 ± 15.8 | 58.0 ± 11.9^{c} | 73.7 ± 42.5 |
| Urea (mmol/L) | 20.0 ± 0.5^{b} | 15.9 ± 1.2^{c} | 14.8 ± 8.6 |
| Pancreatic lipase | 639.8 ± 119.1 | 234.3 ± 33.8 | 456.9 ± 263.8 |
| (U/L) | | | |
| TG (mmol/L) | 0.8 ± 0.2 | 2.5 ± 0.8 | 1.9 ± 1.1 |
| Chol (mmol/L) | 1.9 ± 0.2 | 2.0 ± 0.1 | 2.2 ± 1.3 |
| Glucose (mmol/L) | 13.3 ± 1.6 | 12.8 ± 1.5 | 11.3 ± 6.5 |

LC-FA, blank long chain fatty acid formulation; LC-FA + O, orlistat long chain fatty acid formulation; LFF + O, orlistat lipid free formulation; Na⁺, sodium; K⁺, potassium; Cl⁻, chloride; Ca²⁺, calcium; TP, total protein; CK, creatine kinase; ALT, alanine aminotransferase; AST, aspartate transaminase; ALP, alkaline phosphatase; TG, Triglyceride; Chol, cholesterol. Data is presented as mean ± SEM. Replicates per group were 5 except for acute pancreatitis (AP), lymph diverted, LC-FA + O (n = 3); AP, lymph diverted, LC-FA (n = 6); AP, lymph intact, LFF + O (n = 3).
^{a}Statistically different when compared to sham, lymph diverted rats administered LC-FA + O by one-way ANOVA p ≤ 0.05
^{b}Statistically different when compared to AP, lymph intact rats administered LFF + O by one-way ANOVA p ≤ 0.05
^{c}Statistically different when compared to AP, lymph intact rats administered blank LC-FA formulation by unpaired t test p ≤ 0.05
^{d}Data point is represented as a single data point due to technical errors in the measurement for the other two replicates

TABLE 6
Average blood pressure in the experimental groups during the
0-1.5 h or 1.5-3.5 h post disease induction time period.

| | Sham | Acute pancreatitis | | | | |
|---|---|---|---|---|---|---|
| | Lymph | | | | | |
| | diverted | Lymph diverted | | Lymph intact | | |
| | LC- | | LC- | | LC- | |
| | FA + O | LC-FA | FA + O | LC-FA | FA + O | LFF + O |
| MABP | 93 ± 5 | 80 ± 1^{b} | 99 ± 2 | 84 ± 3 | 96 ± 2^{c} | 95 ± 5 |
| (mmHg) | | | | | | |
| 0-1.5 h | | | | | | |
| MABP | 94 ± 7 | 69 ± 3^{a} | 77 ± 3 | 72 ± 2 | 82 ± 4 | 77 ± 9 |
| (mmHg) | | | | | | |
| 1.5-3.5 h | | | | | | |
| Systolic BP | 118 ± 5 | 104 ± 2^{a} | 119 ± 4 | 115 ± 3 | 125 ± 2 | 120 ± 5 |
| (mmHg) | | | | | | |
| 0-1.5 h | | | | | | |
| Systolic BP | 118 ± 7 | 91 ± 2^{a} | 98 ± 2 | 99 ± 4 | 113 ± 4 | 101 ± 10 |
| (mmHg) | | | | | | |
| 1.5-3.5 h | | | | | | |

LC-FA, blank long chain fatty acid formulation; LC-FA + O, orlistat long chain fatty acid formulation; LFF + O, orlistat lipid free formulation; MABP, mean arterial blood pressure. Data is presented as mean ± SEM. Replicates per group were 5 except for AP, lymph diverted, LC-FA + O (n = 3); AP, lymph diverted, LC-FA (n = 6); AP, lymph intact, LFF + O (n = 3); AP, lymph intact rats administered blank LC-FA (n = 6); and AP, lymph intact rats administered orlistat lipid free formulation (LFF + O). (n = 3).
^{a}Statistically different when compared to sham, lymph diverted rats administered LC-FA + O by one-way ANOVA p ≤ 0.05
^{b}Statistically different when compared to AP, lymph diverted rats administered LC-FA + O by one-way ANOVA p ≤ 0.05

### Example 3. Administration of Self-Emulsifying Drug Delivery System (SEDDS) Enables Mesenteric Lymphatic Transport and Systemic Exposure of Orlistat

### 3.1 Methods

### 3.1.1 Experimental Design

Orlistat solubility was determined in a range of lipid excipients that could be used to prepare a SEDDS formulation including LC-FA (oleic acid, linoleic acid), LC-TG (olive oil), surfactants (Kolliphor EL or cremophor EL, Tween 80, Span 80) and co-solvents (PEG 400) (FIG. 1). This enabled the selection of appropriate excipients to solubilise and formulate the drug.

### 3.1.2 Preparation of Lipid-Based Formulation and Control Formulations

Combinations of LC-FA, surfactant and co-solvent were prepared as type IIIA pre-emulsions (in the combination ratios illustrated in Table 7). The type IIIA formulations were considered the most promising type of SEDDS as they spontaneously form a very fine emulsion on dispersion in water or buffer and contain long-chain lipid, required to promote lymphatic lipid and drug transport. All formulations comprised 8 mg/kg oleic acid. For the control lipid-free formulation, orlistat was dispersed in 112 mg Tween 80 and 5.6 ml PBS.

TABLE 7
Compositions of type IIIA lipid-based formulations tested in the in vitro dispersion and digestion experiments.
PEG 400 polyethylene glycol 400; P35-ECO super refined P35 castor oil, PPG 400 polypropylene glycol 400

| Type IIIA - 1 | | Type IIIA - 2 | | Type IIIA - 3 | | Type IIIA - 4 | | Type IIIA - 5 | |
|---|---|---|---|---|---|---|---|---|---|
| (Tween 80) | | (cremophor EL) | | (span 80) | | (olive oil) | | (P35-ECO) | |
| Excipients | Ratio (%) | Excipients | Ratio (%) | Excipients | Ratio (%) | Excipients | Ratio (%) | Excipients | Ratio (%) |
| Oleic acid | 60 | Oleic acid | 60 | Oleic acid | 60 | Olive oil | 60 | Oleic acid | 40 |
| Tween 80 | 20 | Cremophor EL | 20 | Span 80 | 20 | Tween 80 | 20 | P35-ECO | 50 |
| PEG 400 | 20 | PEG 400 | 20 | PEG 400 | 20 | PEG 400 | 20 | PPG 400 | 10 |

### 3.2 Results

The type IIIA formulations spontaneously produced an emulsion on mixing with water or PBS, and kept the drug mostly solubilised in an oil phase on mixing with simulated intestinal digestion media (FIG. 2). Type IIIA-1 and Type IIIA-5 formulations were chosen for in vivo mesenteric lymph uptake studies in rats where 100 mg of the total formulation containing 8 mg/kg drug and 40-60 mg of oleic acid was dispersed in 5.6 ml PBS and administered to the rats via infusion into the duodenum over 2 hours. These type IIIA formulations were compared to liquid emulsion formulations in which orlistat was dispersed in 40 mg oleic acid, octanoic acid (MC-FA) or olive oil (LC-TG) with 25 mg Tween 80 and 5.6 ml PBS.

Importantly, the concentration of orlistat in lymph over time was almost identical after intestinal administration of the Type IIIA-1 and Type IIIA-5 formulations compared to the liquid emulsion formulations (FIG. 3). Orlistat concentrations in lymph were also well above the IC₅₀ required to inhibit pancreatic lipase. This data supports that oleic acid is able to support lymphatic uptake of orlistat after intestinal/oral administration and that minor changes to the surfactant and co-solvent do not markedly change the lymphatic uptake of the drug. Importantly, these formulations are suitable for preparation of capsules for administration to patients. Alternatively, they may be stored in a vial and mixed with a buffer prior to administration orally or via naso-gastric or naso-jejunal tube.

1. A pharmaceutical formulation comprising a lipase inhibitor and one or more long chain fatty acid.

2. The pharmaceutical formulation according to claim 1, wherein the one or more long chain fatty acid is present in the formulation in an amount of at least 5 wt %.

3. The pharmaceutical formulation according to claim 1, wherein the one or more long chain fatty acid is present in the formulation in an amount of at least 10 wt %.

4. The pharmaceutical formulation according to claim 1, wherein the one or more long chain fatty acid has at least 14 carbon atoms.

5. The pharmaceutical formulation according to claim 1, wherein the one or more long chain fatty is selected from the group consisting of myristic acid, pentadecanoic acid, palmitic acid, palmitoleic acid, heptadecanoic acid, stearic acid, oleic acid, linoleic acid, α-linoleic acid, linolenic acid, stearidonic acid, vaccenic acid, elaidic acid, linolelaidic acid, arachidonic acid, cervonic acid, eicosapentaenoic acid, paullinic acid, gondoic acid, erucic acid, nervonic acid, mead acid, docosatetraenoic acid, docosahexaenoic acid and combinations thereof.

6. The pharmaceutical formulation according to claim 5, wherein the long chain fatty acid is oleic acid.

7. The pharmaceutical formulation according to claim 1, wherein the lipase inhibitor is selected from the group consisting of cetilistat, lipstatin, orlistat, vibralactone, ebelactone, pancilin D, valilactone, esterastin and combinations thereof.

8. The pharmaceutical formulation according to claim 7, wherein the lipase inhibitor is orlistat.

9. The pharmaceutical formulation according to claim 1, further comprising at least one pharmaceutically acceptable carrier, diluent, surfactant or co-solvent.

10. The pharmaceutical formulation according to claim 1, wherein the pharmaceutical formulation is a liquid formulation.

11. The pharmaceutical formulation according to claim 1, wherein the pharmaceutical formulation is a veterinary formulation.

12. A method of treating or preventing a disease or condition mediated by pancreatic lipase in a subject in need thereof comprising administering to the subject an effective amount of the pharmaceutical formulation according to claim 1.

13. A method of treating or preventing acute pancreatitis or an associated syndrome of acute pancreatitis selected from systemic inflammatory response syndrome and/or multiple organ dysfunction syndrome in a subject in need thereof, comprising administering to the subject an effective amount of the pharmaceutical formulation according to claim 1.

14. The method according to claim 12, wherein administration of the pharmaceutical formulation is enteral administration.

15. A method of treating or preventing acute pancreatitis or an associated syndrome of acute pancreatitis selected from systemic inflammatory response syndrome and/or multiple organ dysfunction syndrome in a subject in need thereof, comprising enterally administering to the subject an effective amount of a lipase inhibitor and one or more long chain fatty acid, wherein the one or more long chain fatty acid is present in an amount sufficient to enhance or promote transport of the lipase inhibitor to the intestinal lymph.

16. A method of treating or preventing acute pancreatitis or an associated syndrome of acute pancreatitis selected from systemic inflammatory response syndrome and/or multiple organ dysfunction syndrome in a subject in need thereof, comprising administering to the subject a formulation comprising an effective amount of a lipase inhibitor and one or more long chain fatty acid, wherein the formulation is a self-emulsifying drug delivery system and wherein the one or more long chain fatty acid is present in the formulation in an amount sufficient to enhance or promote transport of the lipase inhibitor to the intestinal lymph.

17. The pharmaceutical formulation according to claim 1 for use in the treatment or prevention of acute pancreatitis and/or an associated syndrome of acute pancreatitis selected from systemic inflammatory response syndrome and/or multiple organ dysfunction syndrome in a subject in need thereof.

18. A method for the treatment or prevention of acute pancreatitis and/or an associated syndrome of acute pancreatitis selected from systemic inflammatory response syndrome and/or multiple organ dysfunction syndrome in a subject in need thereof, the method comprising administering a therapeutically effective amount of a medicament comprising a lipase inhibitor, wherein the lipase inhibitor is formulated with one or more long chain fatty acid and wherein the one or more long chain fatty acid is present in an amount of at least 5 wt %.

19. A method for the treatment or prevention of acute pancreatitis and/or an associated syndrome of acute pancreatitis selected from systemic inflammatory response syndrome and/or multiple organ dysfunction syndrome in a subject in need thereof, the method comprising administering a therapeutically effective amount of a medicament comprising a lipase inhibitor, wherein the lipase inhibitor is formulated with one or more long chain fatty acid and wherein the one or more long chain fatty acid is present in an amount sufficient to enhance or promote transport of the lipase inhibitor to the intestinal lymph when administered.

20. The method according to claim 18, wherein the medicament is a self-emulsifying drug delivery system.

21. The method according to claim 18, wherein the one or more long chain fatty acid has at least 14 carbon atoms.

22. The method according to claim 18, wherein the long chain fatty acid is oleic acid.

23. The method according to claim 18, wherein the lipase inhibitor is orlistat.

24. The method according to claim 13, wherein administration of the pharmaceutical formulation is enteral administration.

25. The method according to claim 19, wherein the medicament is a self-emulsifying drug delivery system.

26. The method according to claim 19, wherein the one or more long chain fatty acid has at least 14 carbon atoms.

27. The method according to claim 19, wherein the long chain fatty acid is oleic acid.

28. The method according to claim 19, wherein the lipase inhibitor is orlistat.
